(19) United States
(12) Patent Application Publication
(10) Pub. No.: US 2026/0215673 A1
(43) Pub. Date: Jul. 30, 2026

(54) LOCAL NAVIGATION AIDS TO ASSIST ENDOSCOPISTS FINDING LOST POLYPS

(71) Applicant: VERILY LIFE SCIENCES LLC, Dallas, TX (US)

(72) Inventors: Natalia Aizenberg, Netanya (IL); Ori Kelner, Tel Aviv (IL); Yuan Zheng, Pacifica, CA (US); Roman Goldenberg, Haifa (IL); Ron Amit, Haifa (IL)

(21) Appl. No.: 19/142,508

(22) PCT Filed: Feb. 12, 2024
(86) PCT No.: PCT/US2024/015414

§ 371 Date: Jun. 23, 2025

Related U.S. Application Data
(60) Provisional application No. 63/487,419, filed on Feb. 28, 2023.

(51) Int. Cl. A61B 1/31; A61B 1/00; G06T 7/73

(52) U.S. Cl. CPC A61B 1/31; A61B 1/000094; A61B 1/000096; A61B 1/0005; G06T 7/73; G06T 2207/10068; G06T 2207/20084; G06T 2207/30032

(57) ABSTRACT

A user-interface for visualizing a colonoscopy procedure includes a video region, a polyp annotation and identification graphic, and a lost polyp navigational aid. A live video feed received from a colonoscope is displayed in the video region. The polyp annotation depicts a location of a polyp within a colon and is presented on the live video feed. When the polyp is lost from the field of view (e.g., as a result of surgical tool insertion), the lost polyp navigation aid displays directions and/or signals to guide the endoscopist back to the lost polyp.

### CROSS-REFERENCE TO RELATED APPLICATIONS

This application claims the benefit of U.S. Provisional Application 63/487,419, filed Feb. 28, 2023, the contents of which are incorporated by reference.

### TECHNICAL FIELD

This disclosure relates generally to endoscopy, and in particular, but not exclusively, to user-interfaces to aid colonoscopy.

### BACKGROUND INFORMATION

When an endoscopist performs a colonoscopy, one of the most important tasks is to ensure that they have visualized every surface of the colon during the procedure to detect all the polyps. On average, between 20% and 24% of polyps that have the potential to become cancerous (adenomas) are missed. Major factors that may cause an endoscopist to miss a polyp are: (1) the polyp appears in the field of view, but the endoscopist misses it, perhaps due to its small size or flat shape; (2) the polyp does not appear in the field of view, as the endoscopist has not fully covered the relevant area during the procedure; and (3) after detecting a polyp, insertion of surgical tools for management of the polyp causes movement of the endoscope and a loss of the area of interest.

Conventional products that assist clinicians/endoscopists with detecting polyps do not currently support features for navigation and return to a lost polyp.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A illustrates a colonoscopy tower system, in accordance with an embodiment of the disclosure.

FIG. 1B illustrates an endoscopy video assistant capable of generating a colonoscopy user-interface including a live video feed and various visual aids during a colonoscopy procedure, in accordance with an embodiment of the disclosure.

FIG. 2 illustrates a colonoscopy user-interface for visualizing a colonoscopy procedure, in accordance with an embodiment of the disclosure.

FIGS. 3A-3D illustrate a navigational map of a colon with coverage annotations and a position marker to aid a colonoscopy procedure, in accordance with an embodiment of the disclosure.

FIG. 4A illustrates how polyps detected within a field of view of the live video feed may be identified and annotated, in accordance with an embodiment of the disclosure.

FIG. 4B illustrates how the colonoscopy user-interface may guide navigation back to a polyp lost from the field of view of the live video feed, in accordance with an embodiment of the disclosure.

FIG. 4C illustrates how the previously lost polyp relocated within the field of view of the live video feed may be annotated, in accordance with an embodiment of the disclosure.

FIG. 5 is a functional block diagram illustrating a demonstrative computing device for implementing an endoscopy video assistant, in accordance with any embodiment of the disclosure.

### DETAILED DESCRIPTION

Embodiments of a system, apparatus, and method for a user-interface (UI) to aid visualization of an endoscopy (particularly colonoscopy) procedure are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Conventional endoscopy interfaces only display the live video feed on the screen without providing any other user aids. Embodiments of the user-interfaces (UI) described herein introduce additional on-screen elements to support and aid the endoscopist in fully visualizing every surface patch of the anatomy being inspected and finding lost points of interest to improve reliability of the overall endoscopy procedure. Although the following description applies aspects of the present disclosure to finding lost polyps in a colon during a colonoscopy procedure, such application is intended as an example, and one skilled in the relevant art will recognize that the embodiments of the technology described herein are suitable for use with any endoscopic procedure and/or with any anatomy, e.g., upper gastrointestinal endoscopy (esophagus, stomach, small intestine), pulmonary endoscopy/bronchoscopy (lungs), laparoscopy, cystoscopy, etc. In further embodiments, the technology described herein can be applied to other imaging fields, such as mechanic/machine inspection endoscopy, self-guided drone imaging, etc.

Deep learning (DL) machine models and techniques may be used in a navigational aid system in accordance with embodiments herein to track directional movements of the colonoscope camera within the colon, for example, after detecting and/or flagging a polyp (e.g., if the camera moves during surgical tool insertion). The directional movements of intubation, withdrawal, lateral, and rotation of the camera can be tracked by the navigational aid embodiments of the disclosure. DL models may further be trained to provide multiple polyp location tracking. Position, depth, and angle tracking along with feature detection and marking (polyp detection and marking) may all be performed based upon image analysis of the video output from the colon. In other embodiments, additional position sensors or real-time scanning techniques may be implemented to obtain position/depth tracking information of the distal end of the colonoscope.

The data obtained from the above image analysis of a live video feed from a colonoscope may be leveraged to display a number of beneficial on-screen visual aids in a colonoscopy UI. These visual aids provide improved operator context and visualization of the colonoscopy procedure. For example, these aids may include a navigational map that depicts longitudinal sections of a colon, a position marker indicating a position of a field of view (FOV) of a camera capturing the live video feed, annotations indicating inspection status of different longitudinal sections of a colon, a cross-sectional coverage map indicating whether portions or surface patches of a longitudinal section have been adequately inspected, guidance arrows prompting the endoscopist back to a recently lost polyp, annotations highlighting detected polyps, and display of a variety of other valuable feedback data (e.g., estimated withdrawal time, polyp detected status, lost polyp navigation, polyp detected history, important notifications, etc.). It should be appreciated that the terms “annotate,” or “annotation” are broadly defined herein to include both textual markups (e.g., on screen textual prompts or dialog) and graphical/pictorial markups (e.g., on screen boxes, arrows, shading, coloring, highlighting, etc.).

Providing these visual aids on the colonoscopy UI in real-time and contemporaneously alongside the live video feed from the colonoscope provides a higher level of context and orientation to the endoscopist. The visual aids increase confidence that all surfaces in the colon have been reviewed and when a polyp is identified, the visual aids provide actionable, real-time feedback to guide the endoscopist back to a lost polyp, e.g., by retracing the directional movement of the colonoscope after identifying and annotating a polyp. Ultimately, the visual aids improve the operator experience thus providing improved tracking of polyps and increased confidence in the overall colonoscopy procedure.

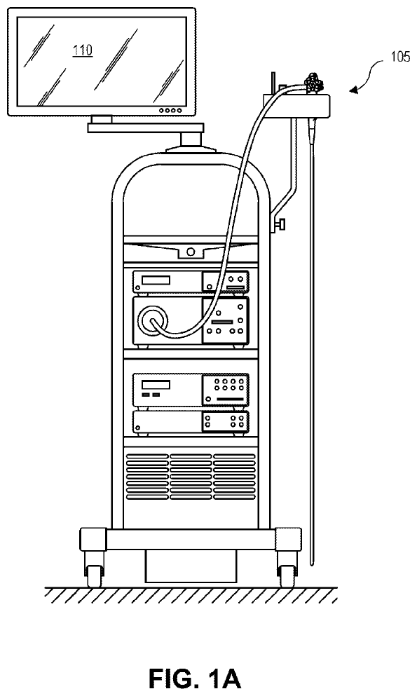

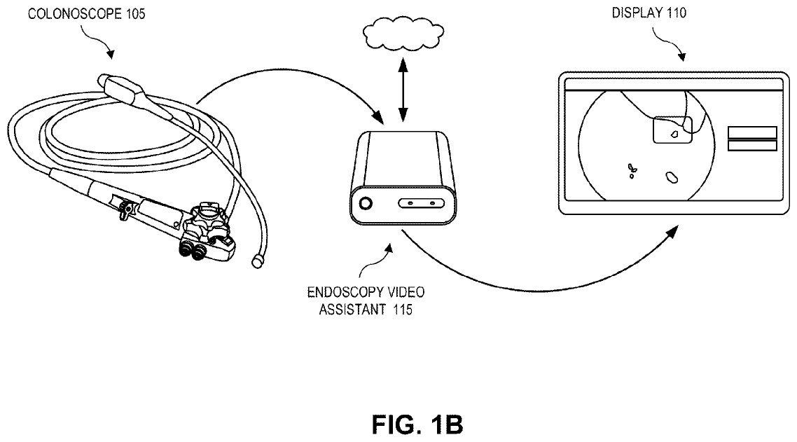

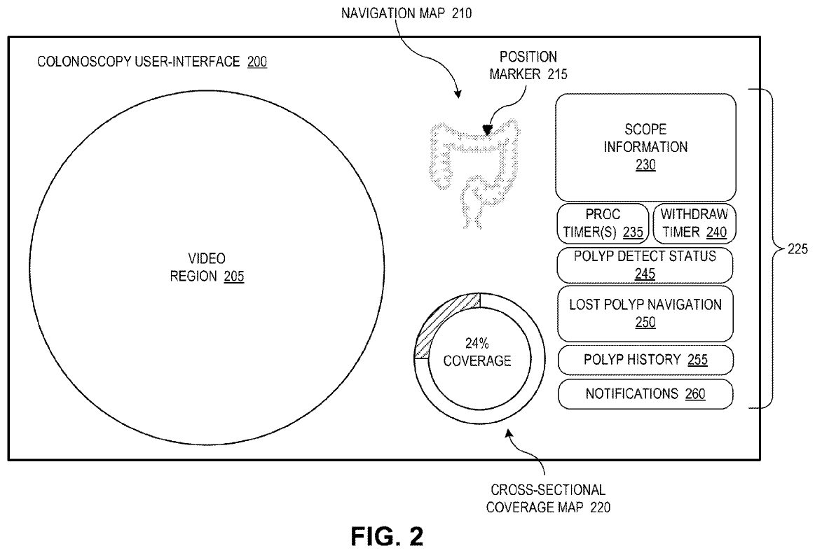

FIG. 1A illustrates a colonoscopy tower system 100, in accordance with an embodiment of the disclosure. The system 100 illustrates an example hardware system in which embodiments of the improved colonoscopy UI described herein may be used. The system 100 includes an endoscope or colonoscope 105 coupled to a display 110 for capturing images of a colon and displaying a live video feed of the colonoscopy procedure. In one embodiment, the image analysis and UI overlays described herein may be performed and generated by a processing box that plugs in between the colonoscope 105 and the display 110. FIG. 1B illustrates an example endoscopy video assistant (EVA) 115 capable of generating the colonoscopy UI described herein. The EVA 115 may include the necessary processing hardware and software, including ML and/or DL models, to perform the real-time image processing and UI overlays. For example, the EVA 115 may include a data storage, a general-purpose processor, graphics processor, and video input/output (I/O) interfaces to receive a live video feed from the colonoscope 105 and output the live video feed within a UI that overlays various visual aids and data. In some embodiments, the EVA 115 may further include a network connection for offloading some of the image processing and/or reporting and saving coverage data for individual patient recall and/or longitudinal, anonymized studies. The colonoscopy UI may include the live video feed reformatted, parsed, or scaled into a video region (e.g., a video region 205 in FIG. 2), or may be a UI overlay on top of the existing colonoscopy monitor feed to maintain the original format, resolution, and integrity of the colonoscopy live video feed as well as reduce any latency.

FIG. 2 illustrates a colonoscopy UI 200 for visualizing a colonoscopy procedure, in accordance with an embodiment of the disclosure. The illustrated embodiment of the colonoscopy UI 200 includes the video region 205 for displaying a live video feed, a navigation map 210 with a position marker 215, a cross-sectional coverage map 220, and a region for procedure data 225. The illustrated embodiment of the procedure data 225 includes scope information 230, procedure timer(s) 235, withdrawal timer 240, polyp detected status 245, lost polyp navigation 250, polyp detected history 255, and notifications 260.

As mentioned, the video region 205 provides a region within the to display a live video feed of the interior of a colon captured during a colonoscopy procedure by a camera of the colonoscope 105. In other words, the video region 205 may be used to display the real-time FOV captured by the camera of the colonoscope 105. Although the video region 205 is illustrated as having a round FOV, in other embodiments, the FOV may be rectangular, square, or otherwise.

The navigation map 210 depicts longitudinal sections of the colon. Each longitudinal section represents a different depth into the colon (or large intestine) extending from the rectum or anal canal to the cecum. The navigation map 210 may be implemented as an anatomical atlas or caricature being representative of the colon, or an actual three-dimensional (3D) model of the colon. In the case of a 3D model, the 3D model of the colon may be generated during an insertion phase of the colonoscopy procedure as the colonoscope 105 is inserted into the anal canal and moved towards the cecum. The live video feed during insertion may be analyzed and mapped into the 3D model. In the illustrated embodiment, the navigation map 210 is annotated with the position marker 215 to indicate a position of the FOV of the live video feed and by extension the distal end of the colonoscope 105 within the colon. In one embodiment, the position marker 215 does not appear on the navigation map 210 until after the colon has been fully mapped or traversed during the insertion phase. After the insertion phase, the position marker 215 moves in real-time tracking the position of the distal end of the colonoscope 105 and the FOV of the live video feed during the withdrawal phase.

The illustrated embodiment of the colonoscopy UI 200 further includes a cross-sectional coverage map 220. The cross-sectional coverage map 220 indicates whether angular portions of a cross-section of a given longitudinal section of the colon is deemed adequately or inadequately inspected. For example, the cross-section coverage map 220 may display a cross-sectional map of the current longitudinal section indicated by the position marker 215. In the illustrated embodiment, the cross-sectional coverage map 220 is indicating that only the surface patch of the colon residing in the upper left quadrant of the current longitudinal section has been adequately inspected and the remaining 76% of the perimeter surface patches of the current longitudinal section have not yet been adequately inspected. During the insertion phase, the image inspection software (e.g., trained neural networks) maps and orients itself to the colon. During the withdrawal phase, the cross-sectional coverage map 220 may map surface patch inspection status relative to the frame of reference of the FOV of the camera during the insertion phase. In other embodiments, the cross-sectional coverage map 220 maps surface patch inspections relative to a current frame of reference or other anatomical reference frames (e.g., sagittal, coronal, or median planes).

The inspection status may be determined or estimated using a combination or weighting of one or more of the following factors: (a) loitering time of a camera of the colonoscope 105 within the given longitudinal section; (b) a determination of whether all surface patches of the colon within the given longitudinal section is observed by the camera (e.g., sweeps within the FOV of the camera for a threshold period of time); (c) a distance between each of the surface patches and the camera when each of the surface patches is observed by the camera; (d) an angle of viewing incidence between the camera and each of the surface patches when each of the surface patches is observed by the camera, or (e) an ML analysis of the colonoscopy video to determine whether any scene potentially included an anatomical fold or area where additional colon anatomy may have be hidden from the FOV.

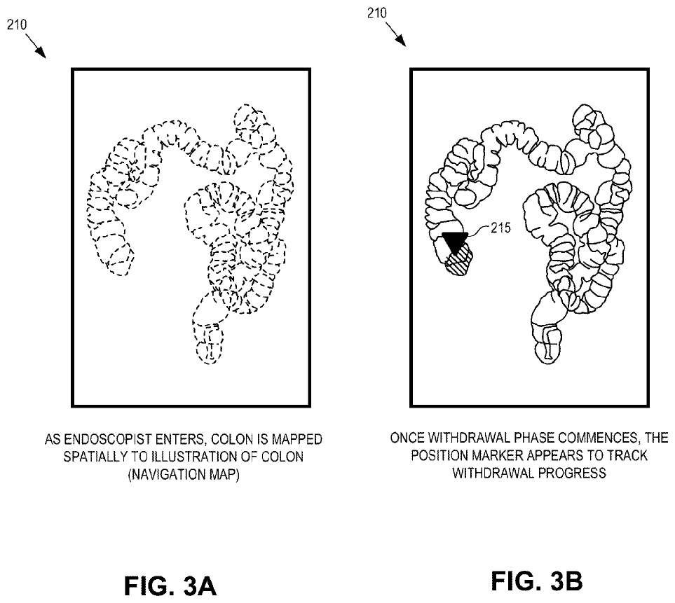

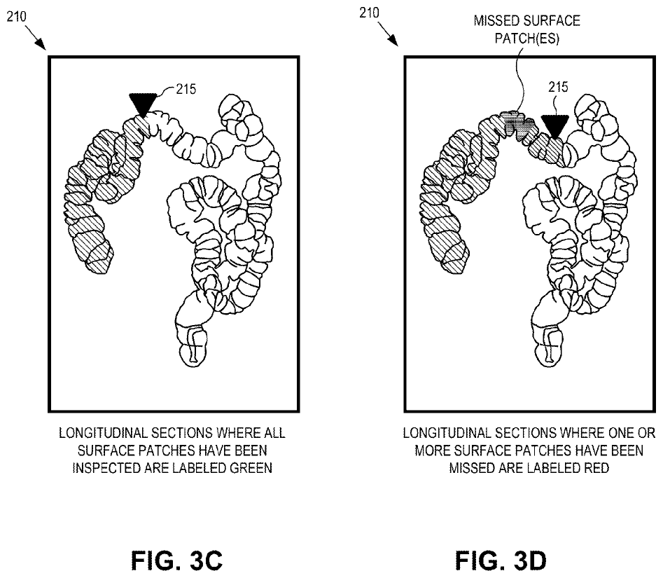

FIGS. 3A-D illustrate further details of the navigational map 210, in accordance with an embodiment of the disclosure. As illustrated in FIG. 3A, the navigational map 210 may be initially presented in a lighter shade or grayed out shade during the insertion phase of the colonoscopy procedure. In yet other embodiments, the navigational map 210 may not be initially presented until the end of the insertion phase or beginning of the withdrawal phase. The insertion phase may be deemed complete once the cecum is reached and recognized as the end of the colon. The colon illustration may be withheld, grayed out, or presented in a lighter shade while the colon is being spatially mapped during the insertion phase. The spatial mapping may be achieved using a 3D visual mapping via image analysis of the live video feed during the insertion phase. In other embodiments, additional sensors and/or tracking devices may be used (alone or in conjunction with the image analysis) to facilitate spatial mapping or generation of a full 3D model of the colon. For example, ultrasound imaging, magnetic tracking, etc. may be used to track the distal tip of the colonoscope 105 as it progresses through the colon.

In FIG. 3B, upon commencement of the withdrawal phase, navigation map 210 is fully presented and position marker 215 displayed. The navigation map 210 along with the position marker 215 present the endoscopist with a visual representation of the position of the FOV of the live video feed within the colon along with a visual estimation of the remaining distance to traverse during the withdrawal phase.

Referring to FIGS. 3C and 3D, as the colonoscope 105 is withdrawn through the colon, the navigation map 210 is annotated to illustrate the inspection status of each longitudinal section along the way. This annotation may be updated in real-time during the withdrawal phase. Longitudinal sections deemed fully inspected (i.e., all surface patches in those longitudinal sections have been adequately inspected) are annotated as such. For example, longitudinal sections that are deemed adequately inspected may be colored green (FIG. 3C). Correspondingly, if the endoscopist withdrawals through a given longitudinal section without fully inspecting every surface patch within that longitudinal section, then the corresponding longitudinal section on the navigation map 210 is annotated to represent an inadequate inspection. For example, the inadequately inspected section may be colored red (FIG. 3D) to indicate that one or more surface patches of the colon in the longitudinal section has been deemed inadequately inspected. Of course, other colors, shades, or labels may be used to indicate adequate or inadequate inspection of a given longitudinal section.

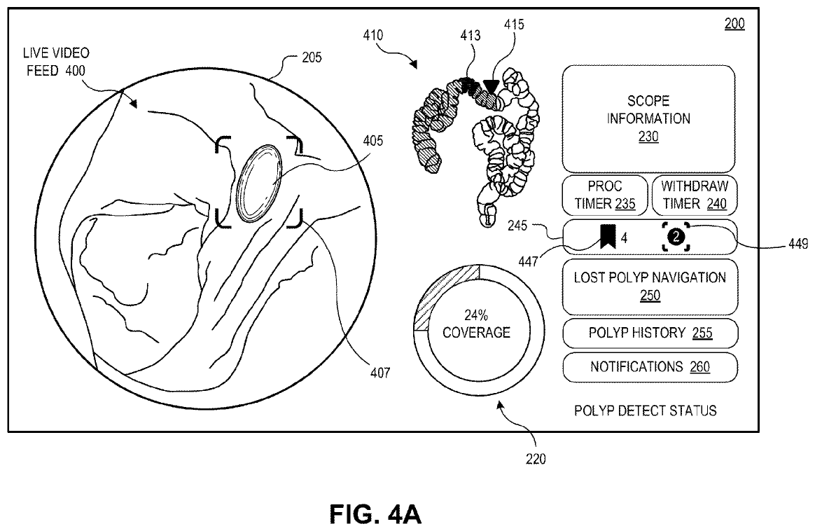

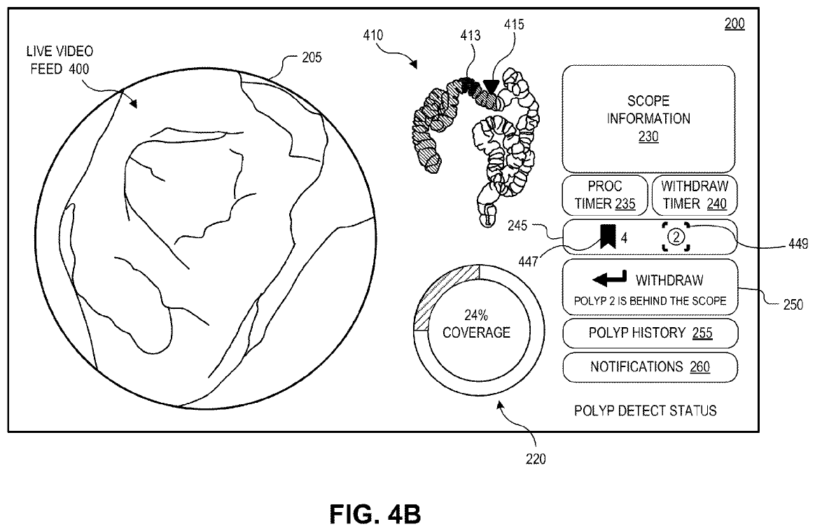

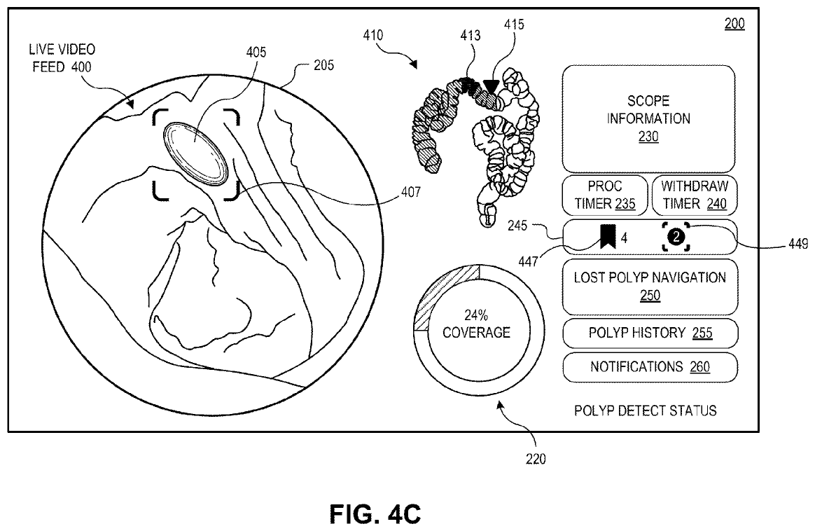

FIG. 4A illustrates how a polyp 405 detected within a field of view of the live video feed 400 may be identified and annotated, in accordance with an embodiment of the disclosure. During the colonoscopy procedure, if a polyp 405 is detected in the live video feed 400, then the detected polyp 405 may be highlighted or accentuated with the annotation 407 clearly identifying its location within the displayed image on the video region 205. As shown, the annotation 407 can overlay the live video feed 400 and move with the identified and marked polyp 405 as the colonoscope 105 moves within the colon. Although FIGS. 4A and 4C illustrate the annotation 407 as corners of a box outline, the annotation may be implemented using a variety of different shapes, colors, shadings, labels, etc.

The polyp detect status 245 represents an indication of whether the image analysis and polyp detect software has detected a polyp in the current live image feed 400 displayed in the video region 205. As polyps are identified, they can be given an identification number such that each polyp can be individually identified. The polyp detect status 245 can display a polyp count graphic 447 showing the total number of polyps given identification numbers. In the illustrated example, the polyp count graphic 447 shows that four polyps have been “bookmarked” to that point in the procedure. When a polyp 405 is detected in the live image feed 400 and marked with the annotation 407, the identification number can be assigned and displayed within the polyp detect status 245 as a polyp identifier graphic 449. In the illustrated example, the polyp 405 shown in the video region 205 is identified as “polyp 2” and when the annotation 407 marks the polyp on the live video feed 400 in the video region 205, the polyp identifier graphic 449 shows a #2 indicating that “polyp 2” is annotated. In the illustrated embodiment, the polyp identifier graphic 449 shows a representative annotation outline with a dark circle having the identification number, which represents a polyp that is tracked and visible within the live video feed 400 on the video region 205. When a polyp is identified, a screenshot or other reference picture of the polyp may be captured for, e.g., review by the endoscopist, reference display on the colonoscopy UI 200, etc.

FIG. 4B illustrates how the colonoscopy user-interface 200 may guide navigation back to a polyp lost from the field of view of the live video feed 400. For example, if a surgical tool is inserted into the colon while the colonoscope 105 is showing “polyp 2” of FIG. 4A, the colonoscope 105 can move to a different location within the colon where “polyp 2” is no longer visible, as shown in the video region 205 of FIG. 4B. In these embodiments, the annotation 407 is removed from the video region 205 and the polyp identifier graphic 449 can change to a light circle having the identification number, which represents a polyp that is tracked but not visible within the live video feed 400 on the video region. Although the polyp identifier graphic 449 is shown with dark and light circles as an indicator of whether the tracked polyp is visible, in other embodiments any suitable indication of the visibility status of the tracked polyp is within the scope of the present disclosure.

When the system 100 detects that the tracked polyp (in the illustrated example, “polyp 2”) is no longer shown in the live video feed 400 of the video region 205, the lost polyp navigation 250 may indicate navigational instructions for the endoscopist to return to the tracked, but visually lost polyp to perform further medical procedures, such as biopsy. Returning to the lost polyp can be performed by retracing the directional movement of the colonoscope since the polyp was last visible in the live video feed 400. In the illustrated example, the colonoscope 105 has traveled further into the colon (intubation) from the location “polyp 2” and needs to be withdrawn to visually relocate the lost polyp. As shown, the lost polyp navigation 250 may display directional cues, such as arrows, text directions (“withdraw,” etc.), positional text (“polyp 2 is behind the scope”), and/or other indicators to guide the endoscopist back to “polyp 2” by retracing the directional movement of the colonoscope 105. In one embodiment, the lost polyp navigation 250 is an aid that can be triggered automatically by additional signals for deducing relevance. For example, if a surgical tool is detected and the identified polyp is not visible, then the system 100 can deduce that the polyp was lost during surgical tool insertion and the endoscopist is interested in the polyp as a result of the tool insertion (e.g. for biopsy). In other embodiments, other additional signals can be used to automatically trigger the lost polyp navigation aid.

The lost polyp navigation aid can use any suitable model for determining the distance, direction, rotation, etc. that the colonoscope 105 has moved since the tracked polyp was visible in the live video feed 400. In some embodiments, deep learning models using computer vision algorithms can infer the movement of the colonoscope 105 since the tracked polyp 405 was lost from the live video feed 400. Deep learning models consist of computer-based learning of classification tasks from images, text, sound, video, etc., and can include algorithms intended to improve accuracy during lost polyp navigation. Among other methods, the lost polyp navigation aid can use one or more of the following techniques: (1) object detection deep neural network, trained for polyp detection: (2) monocular depth and egomotion estimation deep neural networks trained on colonoscopy videos; (3) key-point extraction, matching, and outlier filtering; (4) tool and liquid segmentation module that filters out non-tissue key-points; (5) bundle adjustment optimization that fine tunes the initial estimates to be consistent over time; and/or (6) optical flow in combination with Green theorem or learned models to infer movement direction.

FIG. 4C illustrates how the previously lost polyp 405 relocated within the field of view of the live video feed 400 may be annotated again with the annotation 407. As shown, upon return to the lost polyp, the orientation of the live video feed 400 may be different, e.g., if the colonoscope 105 twisted during movement (compare FIG. 4C to FIG. 4A). When the polyp (e.g., “polyp 2”) returns to the field of view, the lost polyp navigation returns to a neutral state (e.g., no navigational directions displayed) and the polyp identifier graphic 449 shows the dark circle having the identification number, which represents “polyp 2” is tracked and visible within the live video feed 400 on the video region 205. In some embodiments where a screenshot or other image was captured during identification of the polyp, the captured image may be shown as the polyp returns to the field of view such that the endoscopist can compare the captured image with the reidentified polyp to ensure a match. As the procedure continues, the polyp count graphic 447 can continue to indicate the total count of detected polyps and the polyp identifier graphic 449 can indicate which of the identified polyps is currently annotated or being directionally tracked.

Returning to FIG. 2, the colonoscopy UI 200 includes a region for displaying procedure data 225. The illustrated embodiment of the procedure data 225 includes the scope information 230, the procedure timer 235, the withdrawal timer 240, the polyp detected status 245, the lost polyp navigation 250, the polyp history 255, and the notifications 260. The scope information 230 may include metadata pertinent to the particular colonoscope 105 such as camera resolution, software/firmware version, frame rate, color space, etc.

The procedure timer(s) 235 may include one or more timers that track the overall procedure time since commencement of the insertion phase, track the procedure time of just the insertion phase, or track the procedure time since commencement of the withdrawal phase. The withdrawal timer 240 can display an estimated withdrawal time to complete the withdrawal phase of the colonoscopy procedure. The estimated withdrawal time may be calculated using a trained neural network upon inspecting the colon during the insertion phase and may further be updated as the withdrawal phase progresses. As such, the estimated withdrawal time may not be displayed until after completion of the insertion phase and represents a sort of countdown timer until completion of the withdrawal phase.

The polyp history 255 may include a selectable menu for displaying further information regarding the particular detected polyps. For example, if another polyp has been lost during the procedure, selecting the polyp by the identification number can activate the lost polyp navigation aid for the selected polyp, allowing the endoscopist to return to the polyp of interest. In another example, if an ML classifier is applied to perform optical biopsies on the detected polyps, then the results of the optical biopsy may be accessed via the polyp detected history 255 by selecting a given polyp. Alternatively, optical biopsy results and/or reference images for comparison may automatically appear when a polyp is identified in the FOV. The results may include a classification of benign, precancerous, cancerous, etc. along with display of a confidence interval. Finally, procedure data 225 may further include a section for notifications 260 where miscellaneous notifications including polyp types/classifications may also be presented.

Embodiments disclosed herein provide the colonoscopy UI 200 that contemporaneously presents the live video feed from the colonoscope 105 alongside contextual/orientational data from the navigation map 210, the cross-sectional coverage map 220, and the procedure data 225. These contemporaneous visual aids provide a higher level of context and orientation to the endoscopist, thereby improving the reliability of the colonoscopy procedure and confidence that all polyps are detected.

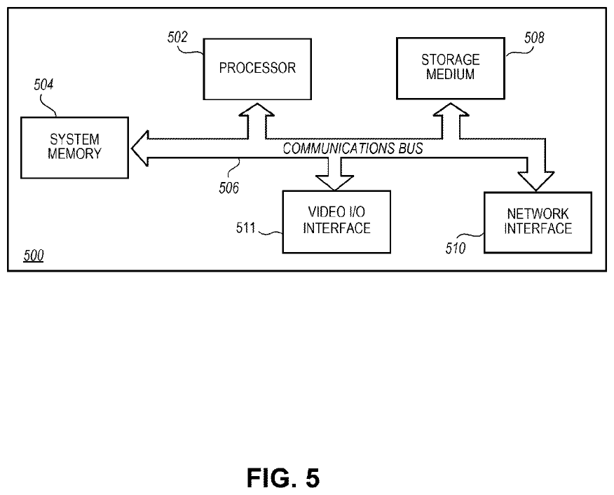

FIG. 5 is a block diagram that illustrates aspects of a demonstrative computing device appropriate for implementing the EVA 115, in accordance with embodiments of the present disclosure. Those of ordinary skill in the art will recognize that the computing device 500 may be implemented using currently available computing devices or yet to be developed devices.

In its most basic configuration, the computing device 500 includes at least one processor 502 and a system memory 504 connected by a communication bus 506. Depending on the exact configuration and type of device, the system memory 504 may be volatile or nonvolatile memory, such as read only memory (“ROM”), random access memory (“RAM”), EEPROM, flash memory, or similar memory technology. Those of ordinary skill in the art will recognize that the system memory 504 typically stores data and/or program modules that are immediately accessible to and/or currently being operated on by the processor 502. In this regard, the processor 502 may serve as a computational center of the computing device 500 by supporting the execution of instructions.

As further illustrated in FIG. 5, the computing device 500 may include a network interface 510 comprising one or more components for communicating with other devices over a network. Embodiments of the present disclosure may access basic services that utilize the network interface 510 to perform communications using common network protocols. The network interface 510 may also include a wireless network interface configured to communicate via one or more wireless communication protocols, such as WiFi, 2G, 3G, 4G, LTE, 5G, WiMAX, Bluetooth, and/or the like.

In the exemplary embodiment depicted in FIG. 5, the computing device 500 also includes a storage medium 508. However, services may be accessed using a computing device that does not include means for persisting data to a local storage medium. Therefore, the storage medium 508 may be omitted. In any event, the storage medium 508 may be volatile or nonvolatile, removable or nonremovable, implemented using any technology capable of storing information such as, but not limited to, a hard drive, solid state drive, CD-ROM, DVD, or other disk storage, magnetic cassettes, magnetic tape, magnetic disk storage, and/or the like.

The illustrated embodiment of the computing device 500 further includes a video input/out interface 511. The video I/O interface 511 may include an analog video input (e.g., composite video, component video, VGG connector, etc) or a digital video input (e.g., HDMI, DVI, DisplayPort, USB-A, USB-C, etc.) to receive the live video feed from the colonoscope 105 and a similar type of video output port to output the live video feed within the colonoscopy UI 200 to the display 110. In one embodiment, the video I/O interface 511 may also represent a graphics processing unit capable of performing the necessary computational video processing to generate and render the colonoscopy UI 200.

As used herein, the term “computer-readable medium” includes volatile and non-volatile and removable and non-removable media implemented in any method or technology capable of storing information, such as computer-readable instructions, data structures, program modules, or other data. In this regard, the system memory 504 and the storage medium 508 depicted in FIG. 5 are merely examples of computer-readable media.

Suitable implementations of computing devices that include a processor 502, system memory 504, communication bus 506, storage medium 508, and network interface 510 are known and commercially available. For ease of illustration and because it is not important for an understanding of the claimed subject matter, FIG. 5 does not show some of the typical components of many computing devices. In this regard, the computing device 500 may include input devices, such as a keyboard, keypad, mouse, microphone, touch input device, touch screen, tablet, and/or the like. Such input devices may be coupled to the computing device 500 by wired or wireless connections including RF, infrared, serial, parallel, Bluetooth, USB, or other suitable connection protocols using wireless or physical connections. Since these devices are well known in the art, they are not illustrated or described further herein.

The above user-interface has been described in terms of a colonoscopy and is particularly well-suited as a colonoscopy user-interface to aid visualization of colonoscopy procedures. However, it should be appreciated that the user-interface 200 may be more broadly/generically described as an endoscopy user-interface that may be used to visualize endoscopy procedures, in general, related to other anatomical structures. For example, the user-interface is applicable to aid visualization of other gastroenterological procedures including endoscopy procedures within the upper and lower gastrointestinal tracts. In yet other examples, the user-interface may be used to visualize exploratory endoscopy procedures of non-gastroenterological structures such as the esophagus, bronchial tubes, other tube-like anatomical structures, etc. When adapting the user-interface to visualize other endoscopy procedures, the navigational map 210 would represent a map of the corresponding anatomical structure being explored and the cross-sectional coverage map 220 would represent cross-sectional or perimeter inspection coverage of the corresponding anatomical structure, and can be adapted to different tissue types, tumors, etc. to support clinical workflow related to other endoscopy procedures.

The processes and user-interface described above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, some of the processes or logic for implementing the user-interface may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. At least one machine-accessible storage medium that provides instructions that, when executed by a machine, will cause the machine to perform operations comprising:
generating a colonoscopy user-interface for display on a screen,
outputting a live video feed received from a colonoscope for display within a video region of the colonoscopy user-interface;
annotating a polyp on the live video feed within the video region to visually indicate a location of the polyp with an annotation of the polyp;
tracking a directional movement of the colonoscope after annotating the polyp; and
after the polyp is no longer visible on the live video feed within the video region, generating a lost polyp navigation aid for display within the colonoscopy user-interface to guide the colonoscope back to the polyp, wherein the lost polyp navigation aid indicates a guide direction to retrace the directional movement of the colonoscope based upon tracking the directional movement.

2. The at least one machine-accessible storage medium of claim 1, wherein the live video feed and the lost polyp navigational aid are both contemporaneously presented within the colonoscopy user-interface.

3. The at least one machine-accessible storage medium of claim 1, wherein the annotation of the polyp is automatic when the polyp is displayed on the live video feed.

4. The at least one machine-accessible storage medium of claim 1, wherein the annotation of the polyp is removed from the video region when the polyp is no longer displayed on the live video feed.

5. The at least one machine-accessible storage medium of claim 1, further providing instructions that, when executed by the machine, will cause the machine to perform further operations, comprising:
assigning an identification number to the polyp contemporaneously with the annotation; and
outputting the polyp identification number for display within a procedure data region of the colonoscopy user-interface.

6. The at least one machine-accessible storage medium of claim 5, wherein outputting the polyp identification number comprises outputting a polyp identifier graphic indicative of whether the polyp is visible on the live video feed within the video region.

7. The at least one machine-accessible storage medium of claim 5, further providing instructions that, when executed by the machine, will cause the machine to perform further operations, comprising:
outputting a polyp count graphic indicative of the total number of polyps assigned an identification number to the colonoscopy user-interface.

8. The at least one machine-accessible storage medium of claim 1, further providing instructions that, when executed by the machine, will cause the machine to perform further operations, comprising:
detecting return of the polyp on the live video feed; and
reannotating the polyp on the live video feed within the video region to visually indicate the location of the polyp.

9. The at least one machine-accessible storage medium of claim 8, further providing instructions that, when executed by the machine, will cause the machine to perform further operations, comprising:
capturing an image of the polyp contemporaneously with the annotation; and
outputting the captured image of the polyp for display within the colonoscopy user-interface after reannotating the polyp on the live video feed to permit visual comparison between the reannotated polyp and the captured image.

10. The at least one machine-accessible storage medium of claim 1, wherein generating the lost polyp navigation aid further comprises generating directional indication arrows, directional text, positional text, or a combination thereof.

11. The at least one machine-accessible storage medium of claim 1, wherein generating the lost polyp navigation aid is automatic when a tool is present in the live video feed and the polyp is no longer visible on the live video feed within the video region.

12. At least one machine-accessible storage medium that provides instructions that, when executed by a machine, will cause the machine to output a signal for rendering a user-interface to a display, the user-interface adapted for visualizing an endoscopy procedure, the user-interface comprising:
a video region in which a live video feed received from an endoscope is displayed;
an annotation of a polyp on the live video feed within the video region to visually indicate a presence of the polyp; and
a lost polyp navigation aid within the colonoscopy user-interface to guide the colonoscope back to the polyp after the polyp is no longer visible on the live video feed within the video region,
wherein the lost polyp navigation aid indicates a guide direction to retrace a directional movement of the colonoscope occurring since the polyp was last visible on the live video feed within the video region.

13. The least one machine-accessible storage medium of claim 12, wherein the user-interface further comprises:
an identification number of the polyp displayed within a procedure data region of the colonoscopy user-interface.

14. The least one machine-accessible storage medium of claim 12, wherein the user-interface further comprises:
a polyp identifier graphic displayed within the colonoscopy user-interface and indicative of whether the polyp is currently visible on the live video feed within the video region.

15. The least one machine-accessible storage medium of claim 12, wherein the user-interface further comprises:
a polyp count graphic displayed within the colonoscopy user-interface and indicative of the total number of polyps assigned an identification number.

16. The least one machine-accessible storage medium of claim 12, wherein the user-interface further comprises:
a reannotation of the polyp on the live video feed within the video region to visually indicate the presence of the reannotated polyp.

17. The least one machine-accessible storage medium of claim 16, wherein the user-interface further comprises:
a captured image of the polyp captured during annotating the polyp, the captured image displayed after the reannotation of the polyp on the live video feed to permit visual comparison between the reannotated polyp and the captured image.

18. The least one machine-accessible storage medium of claim 12, wherein the user-interface further comprises:
directional indication arrows, directional text, positional text, or a combination thereof displayed within the colonoscopy user-interface and indicative of a guide direction retracing the directional movement of the colonoscope after annotation of the polyp.

19. The least one machine-accessible storage medium of claim 12, wherein the user-interface further comprises:
automatic display of the lost polyp navigation aid when a tool is present in the live video feed and the polyp is no longer visible on the live video feed within the video region.

20. The least one machine-accessible storage medium of claim 12, wherein the user-interface comprises a colonoscopy user-interface, the endoscopy procedure comprises a colonoscopy procedure, the endoscope comprises a colonoscope, and the polyp comprises a polyp on a colon.
